(19) United States
(12) Patent Application Publication
(10) Pub. No.: US 2009/0036859 A1
(43) Pub. Date: Feb. 5, 2009

(54) Anthroprometrically expandable tampon pledget

(75) Inventors: Eugene Dougherty, JR., Camden-Wyoming, DE (US); Keith Edgett, Middletown, DE (US); Shannon Talbot, Newark, DE (US); Robert Jorgensen, Middletown, DE (US)

Correspondence Address: MICHAUD-DUFFY GROUP LLP, 306 INDUSTRIAL PARK ROAD, SUITE 206, MIDDLETOWN, CT, 06457, US

(73) Assignee: Playtex Products, Inc., Westport, CT (US)

(21) Appl. No.: 11/890,011

(22) Filed: Aug. 3, 2007

(51) Int. Cl. A61F 13/22

(52) U.S. Cl. 604/385.18; 264/103

(57) ABSTRACT

A tampon pledget has an absorbent mass of material and a withdrawal string located at one end thereof. During use, the tampon pledget expands to take on a geometric configuration that approximates the shape of the vagina into which the tampon pledget is inserted. An anthroprometrically expandable tampon pledget has an absorbent mass of material that is defined by a first end having a first density, a second end having a second density, and a portion intermediate the first and second ends. The material expands at different rates upon being contacted by body fluids, thereby allowing the tampon pledget to conform to the shape of a vagina. A method of making an expandable tampon pledget includes the steps of providing an absorbent material, attaching a string thereto, and compressing the material into a cylindrical shape such that a density gradient is realized over a length of the compressed material.

### TECHNICAL FIELD

The present invention generally relates to tampon pledgets and, more particularly, to tampon pledgets that expand in the vaginal canal in response to the absorption of body fluids.

### BACKGROUND OF THE INVENTION

In female placental mammals, particularly humans, the vagina is an elastic muscular canal that extends from the uterus inside the body to the vulva on the outside the body. The narrow lower neck of the uterus forms the cervix. The entrance to the vagina is known as the introitus and is located at the back end of the vulva.

Although there is wide anatomical variation, the average length of the canal from the introitus to the cervix is about 3 to 5 inches. The introitus is of a relatively small diameter, and the bottom two-thirds of the vagina is only slightly, if at all, wider than the introitus. The transverse (side-to-side) width in the bottom two-thirds portion is typically slightly larger than the front-to-back width, which is especially small. The vagina is widest in its upper one-third portion nearest the cervix. The elasticity of the walls of the canal that forms the vagina allows it to stretch as needed. When the vagina is in a relaxed state, the walls thereof typically collapse on each other but can be opened up slightly with minimal pressure.

From a biological perspective, the vagina provides a path for fluids to leave the body during the menstruation phase of the menstrual cycle. The menstrual cycle is a recurring cycle of physiological changes in females that is associated with reproductive fertility. The menstruation phase of the menstrual cycle involves the shedding of the uterine lining. During this shedding, tissue and blood is exuded from the uterus.

In modern cultures, disposable absorbent devices have been used for the capture and absorption of material, particularly the blood, exuded during the menstruation phase. These devices include external-use sanitary towels or napkins and internal-use devices such as tampon pledgets. Other devices such as internally-worn cups are also known but not in wide use today.

Tampon pledgets generally comprise non-woven webs of absorbent materials such as rayon, cotton, combinations of the foregoing, and/or other materials in fiber form. These webs are typically stacked such the interfacial areas thereof are bonded together. The stacked and bonded webs are then folded, pleated, and/or rolled to approximate the desired geometry, which is typically a generally cylindrical shape. One end of a string is incorporated into the web material, which is then compressed to a final shape. The string functions as a means of withdrawing the tampon pledget from the vagina after the useful life of the tampon pledget. A coverstock may be located over the compressed absorbent materials to maintain the cylindrical shape and/or to provide increased comfort during the insertion and removal of the tampon pledget. The tampon pledgets are then optionally inserted into a cardboard or plastic applicator device and packaged.

When inserted into the vagina and expanded, such conventional tampon pledgets are generally sufficient to accommodate differences in menstrual flow patterns for the vast majority of women. However, the differences in anatomical structure of women oftentimes results in a less than optimal conformance to the vaginal shape. When the tampon pledget does not conform to the vaginal shape, the likelihood of flow of body fluids from the cervix, around the tampon, and to the introitus is increased. Many conventional tampon pledgets generally conform to the shape of the vagina. However, the shapes of such pledgets are determined and attained prior to contact of the material by the body fluids.

Based on the foregoing, it is an object of the present invention to provide a tampon pledget that expands when contacted by body fluids to more closely approximate the shape of the vagina to prevent or at least minimize the likelihood of leakage of body fluids from the vagina.

### SUMMARY OF THE INVENTION

In one aspect, the present invention resides in a tampon pledget having an absorbent mass of material and a withdrawal string located at one end thereof. The tampon pledget is initially cylindrical in shape. During use and upon contact with menses or other body fluids, the tampon pledget expands to take on a geometric configuration that approximates the shape and configuration of the vagina into which the tampon pledget is inserted. This expansion results largely from the density distribution of the materials utilized to form the tampon pledget. Preferably, the tampon pledget expands to form an hourglass-shaped member that provides suitable contact with the vagina walls. The upper portion of the hourglass shape absorbs the bulk of the menses or other body fluid, while the middle portion and the lower portion proximate the introitus also contact the walls of the vaginal cavity to absorb any menses or other fluid that may have bypassed the upper portion.

In another aspect, the present invention resides in an anthroprometrically expandable tampon pledget having an absorbent mass of material that is defined by a first end having a first density, a second end having a second density, and a portion intermediate the first and second ends. This intermediate portion has a third density that is different from at least one of the first density and the second density. The differences in densities allow the material of the mass to expand at different rates upon being contacted by body fluids, thereby allowing the tampon pledget to conform to the shape of a vaginal cavity. The tampon pledget also has means for withdrawing the tampon pledget from the vaginal cavity (such as a string). The absorbent mass of material is cylindrical in shape.

In another aspect, the present invention resides in a method of making an expandable tampon pledget. This method includes the steps of providing at least one absorbent material, attaching a string thereto, and compressing the absorbent material into a final cylindrical shape such that a density gradient is realized over a length of the compressed absorbent material. A density gradient defined along the length of the tampon pledget allows it to anthroprometrically expand to the shape of a vaginal cavity into which it is inserted upon contact with body fluid.

Although the conformance of the shape of the tampon pledget to the vaginal cavity allows for the absorption of fluid while minimizing leaking to the introitus, several other advantages will become apparent. First, such a configuration provides optimal comfort to the user. In particular, because the tampon pledget expands to contact the walls of the vagina, it exerts pressure in the radial directions, thereby allowing the tampon pledget to be securely held in the vaginal canal. This provides an improved level of comfort to the user, particularly in instances in which the user is physically active.

Second, the cylindrical shape of the tampon pledget is relatively inexpensive and easy to manufacture. Tooling apparatus and manufacturing processes, particularly those related to compressing the pledget material into the pre-expanded shape, can be simple in design.

Third, because the pledgets of the present invention are initially cylindrical rather than curved or serpentine in shape, only a minimal amount of force is required to eject these tampon pledgets from convention tampon pledget applicators (which are typically cardboard or plastic). Thus, the tampon pledgets of the present invention may be used with existing applicators, and no new tooling or design considerations are necessary.

These and other advantages of the present invention will become apparent to those of skill in the art from the following description of the preferred embodiments.

### DESCRIPTION OF THE DRAWINGS

FIG. 1 is a schematic representation of a tampon pledget of the present invention.

FIG. 2 is a schematic representation of the tampon pledget of FIG. 1 in an expanded state.

FIG. 3 is a schematic representation of a first rolled web around the ends of which second webs are rolled to form an absorbent mass of a tampon pledget of the present invention.

FIG. 4 is a schematic representation of layers of webs of alternating sizes for subsequent rolling to form an absorbent mass of a tampon pledget of the present invention.

FIG. 5 is a perspective view of a tampon pledget of the present invention.

FIG. 6 is a graphical representation illustrating how various portions of the tampon pledget of the present invention change during use to anthroprometrically conform to the shape of a vaginal canal.

FIG. 7A is an MRI scan of a tampon pledget in a vagina.

FIG. 7B is an MRI scan of a tampon pledget in a vagina.

FIG. 7C is an MRI scan of a tampon pledget in a vagina.

FIG. 8 is a perspective view of a cone-shaped tampon pledget of the present invention.

FIG. 9 is a perspective view of a serpentine-shaped tampon pledget of the present invention.

### DESCRIPTION OF THE PREFERRED EMBODIMENTS

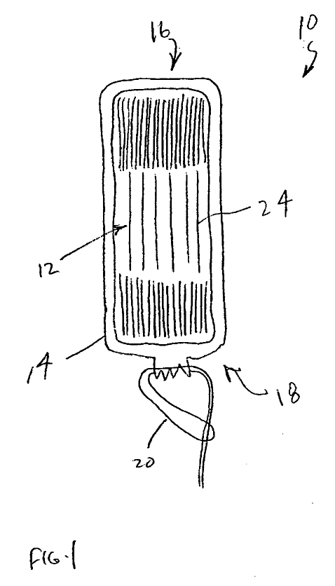

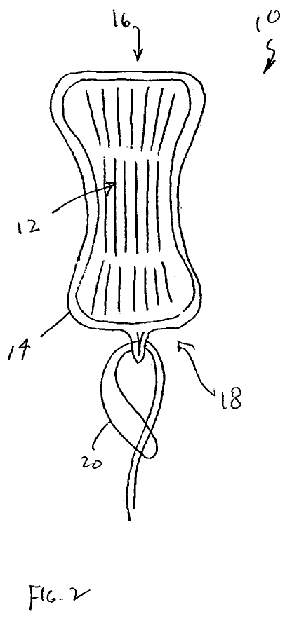

As shown in FIGS. 1 and 2, a tampon pledget of the present invention is generally designated by the reference numeral 10 and is hereinafter referred to as “pledget 10.” The pledget 10 includes an absorbent mass 12 of suitable material. An insertion end 16 is located at one end of the absorbent mass 12, and a withdrawal end 18 is located at an opposing end of the absorbent mass. A removal string 20 is incorporated into the absorbent mass 12 proximate the withdrawal end 18. The absorbent mass 12 may or may not be sheathed in a coverstock 14 that is capable of allowing the flow of menses or other body fluid therethrough. If the coverstock 14 is included, the removal string 20 can be tied thereto proximate the withdrawal end 18.

In its initial form (FIG. 1), the pledget 10 is cylindrical in shape. During use, the pledget 10 expands in response to the absorption of menses or other body fluids to an anthroprometric geometry (FIG. 2) in order to approximate the shape of a vaginal cavity into which the pledget is inserted. As used herein, the phrase “anthroprometric” is intended herein to indicate expandable upon contact with fluid to conform to a bounded area.

Referring specifically to FIG. 1, the absorbent mass 12 is defined by an absorbent material. This absorbent material is generally rayon, cellulosic material such as cotton or paper, or combinations of the foregoing, although other materials are within the scope of the present invention. In one embodiment, the absorbent material is defined by one or more webs 24. These webs 24 are woven strands of material. In the alternative, the webs 24 may be open-cell foam or sponge. The present invention is not limited to woven strands of material, foam material, or sponge, however, as other forms of the materials such as felts or other non-stranded materials may be used.

The webs 24 are arranged such that about one third of the absorbent mass 12 proximate the insertion end 16 of the finished pledget 10 has an initial predetermined density. The webs 24 are also arranged such that about one third of the absorbent mass 12 proximate the withdrawal end 18 of the finished pledget 10 also has an initial predetermined density. These two densities, which may or may not be the same, are based on the mass of absorbent material per unit volume and at least one of the particular geometry of the web material used, the method of constructing the pledget 10, the application of pressure to the absorbent mass 12 to form it to the cylindrical shape, the direction and duration of pressure application, and ambient temperature.

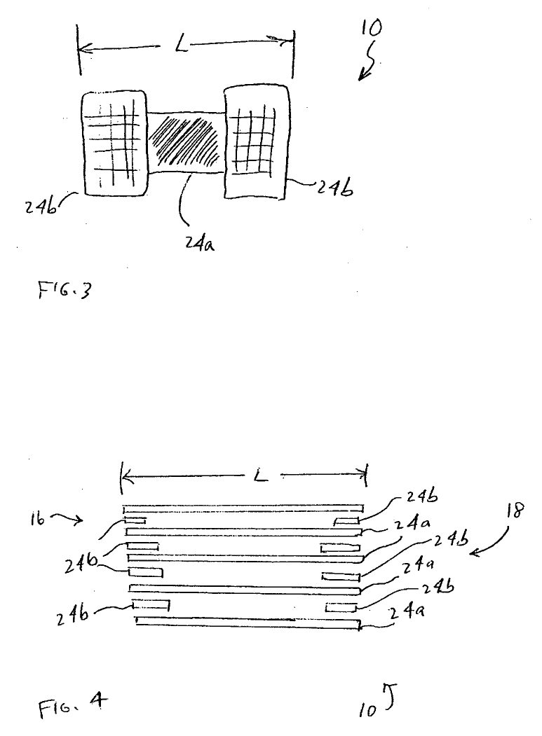

In one embodiment as shown in FIG. 3, the absorbent mass 12 is arranged by combining webs of varying sizes into an approximate initial cylindrical shape. The webs are combined by rolling a first web 24a into a cylindrical shape that substantially corresponds to a length L of the pledget 10 and rolling end webs 24b around the ends of the rolled first web 24a. The present invention is not limited to the rolling of webs, as other means of combining the materials (such as folding, gluing, and the like) are within the scope of the present invention. Furthermore, the present invention is not limited to the use of two or more webs, as one web may be rolled or folded to form the approximate initial cylindrical shape.

In another embodiment as shown in FIG. 4, the webs are combined by rolling a plurality of web materials of different sizes. In particular, a plurality of first webs 24a that substantially correspond to the length L of the finished pledget are interwound with a plurality of second webs 24b that are shorter in a lengthwise dimension and selectively placed proximate the insertion end 16 and the withdrawal end 18 of the finished pledget 10.

The present invention is not limited to the combination of webs of varying sizes, as webs of varying shapes and weights may also be used to form the tampon pledget of the present invention.

In any embodiment, the webs can be produced by combing or carding using a suitable combing or carding device to produce a matte of non-woven fibers. Fibers in this matte can be oriented in a particular direction by, for example, a carding machine and aligned. In the alternative, the fibers can be randomly arranged. By varying the number, distribution, and orientation of absorbing fibers relative to non-absorbing fibers, the density of the matte can be adjusted.

The fibers can be bound together using any of a variety of techniques. For example, the fibers can be bound using barbed needles in a needletacking process; they can be bound chemically using adhesives, water-dispersible binders, or the like; they can be bound thermally using high temperatures; or they can be bound using a hydroentangling technique using high-pressure water jets. Irrespective of the technique used, the density of the matte can be adjusted during the particular technique. The binding generally prevents the sloughing of fibers in the woman's vagina. In the alternative, the fibers in the matte can be left unbound, and a second web of non-woven material can be used as a covering to prevent the sloughing of unbound fibers.

Once the matte has been finally formed, a portion of material is cut therefrom, wound into the approximate shape of a tampon, and cut to the size desired for a specific absorbency range. The material can be rolled up using mechanical means in which the material is wound on itself, or it can be folded into a desired shape (e.g., a “W” shape or the like) using folding rams. Typically, after either rolling or folding, the tampon is compressed into a self-sustaining cylindrical form. In addition to compressive forces, temperature and moisture content may be adjusted to provide the desired cylindrical form. The density of the tampon can also be adjusted by applying compressive forces of differing amounts of pressure. Once in the cylindrical form, the tampon can be combined with a tampon applicator.

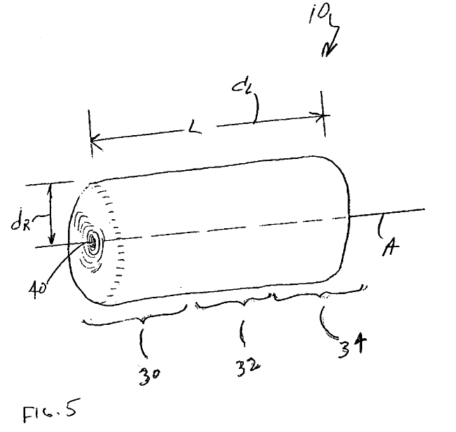

Referring now to FIG. 5, once the webs are rolled into the absorbent mass 12 that approximates an initial cylindrical shape, they are compressed into a substantially uniform final cylindrical geometry. Compression of the absorbent mass 12 generally occurs in one or more radial directions to define the sides of the pledget 10. Compression may also occur in one or both axial directions to define the ends of the pledget 10. In either case, suitable tooling is used to define the surfaces of the pledget 10.

Before the absorbent mass 12 is compressed, a top third portion 30 thereof has a greater initial mass and volume of absorbent material than does a middle third portion 32. When the absorbent mass 12 is compressed (FIG. 5 shows the pledget 10 after compression), both the top third portion 30 and the middle third portion 32 have the same final volume of absorbent material, but the absorbent material of the top third portion is more tightly compressed. The top third portion 30 is therefore denser than the middle third portion 32. A bottom third portion 34 also has a greater initial mass and volume of absorbent material than the middle third portion 32 and, when compressed to the same final volume as the middle third portion, is denser than the middle third portion. Although this disclosure refers to the portions of the absorbent mass as “thirds,” it should be understood that each “third” is not required to be of equal size. Compression of the absorbent material also affects both absorption capacity, i.e., the volume of fluid absorbed, as well as the absorbency rate, i.e., how fast menses or other body fluids are taken up by the tampon. Both affect the expansion characteristics of the pledget 10 during the intake of menses or other body fluids during use.

After compression in which the top third portion 30 and the bottom third portion 34 are compressed to correspond in cross sectional geometry to the middle third portion 32, the density varies along the length of the pledget 10 to produce a lengthwise density gradient d_{L}. This lengthwise density gradient d_{L} enables different portions of the pledget 10 to absorb fluid at different rates and therefore to expand at different rates. In particular, the denser top third portion 30 has a greater potential porosity than the middle third portion 32 and therefore has the potential to expand to a volume that is greater than the volume of the middle third portion 32. The bottom third portion 34, which also includes more tightly compressed and therefore denser absorbent material, expands in a manner similar to the top third portion 30.

In embodiments in which web materials for the top third portion 30 and the bottom third portion 34 differ from the material of the first web (shown as 24a in FIG. 4), a core 40 is defined at the center of the rolled webs and along an axis A. In such an embodiment, the density may be different near the external surfaces of the pledget 10, thereby producing a radial density gradient d_{R}. Also in such an embodiment, the radial density gradient d_{R} may be different at various points along the axis A, particularly in the middle third portion 32. A compression tool or a crimping tool may be utilized to compress or crimp the pledget 10 at various points along the length thereof, thereby causing the radial density gradient d_{R} to be different at the various points along the pledget.

Irrespective of whether or not the pledget 10 includes a radial density gradient d_{R}, the geometry of the web material(s), the particular method of construction of the pledget, the application of pressure, the directions (radial and/or axial) of pressure application, the duration of pressure application, and the temperature are all factors in the determination of the ability of the pledget to absorb menses or other body fluids. Collectively, these factors are used to define a density profile of the pledget 10.

Once the absorbent mass 12 is compressed into its desired cylindrical form, the coverstock, if desired, can then be located thereover.

Referring back to FIG. 2, the anthroprometric geometry of the pledget 10 during use is shown. As menses or other body fluids enter the pledget 10, the density profile of the pledget is altered. In particular, as menses and other body fluids are absorbed, variations in the number of pores, pore sizes, and pore size distributions throughout the material of the pledget 10 effect changes in the capillary pressure throughout the pledget itself. These changes in capillary pressure influence the absorption of the fluid considerably, namely, by causing the pledget 10 to expand to the anthroprometric geometry. Particularly because of the differences in density along the length of the pledget 10, the anthroprometric expansion of the absorbent mass 12 causes the pledget to expand into the shape of the vagina. Preferably, the pledget 10 expands to form an hourglass-shaped object, i.e., wide at the insertion end 16 where the pledget is closest to the cervix, narrower in the middle where the vagina narrows, and slightly widened at the withdrawal end 18 near the introitus. When the pledget 10 is constructed as described above, this expansion occurs rapidly. The hourglass shape develops from the initial onset of fluid and persists until the pledget 10 is removed from the vagina.

The actual preferred anthroprometric geometry and density profile of the pledget 10 during use depends on several factors. Thus, the particular embodiments of the pledget 10 described herein are not limited to those shown. Factors that may contribute to the anthroprometric geometry of the manufactured pledget 10 and its density profile during use include, but are not limited to, the geometry of the particular vagina in which the pledget is inserted, the absorbency of the material used to construct the pledget, the need for improved leakage protection such as bypass protection, and the need for comfort during the insertion, wearing, and removal of the pledget. Thus, the precise quantitative parameters for optimum geometries and densities can vary. More specifically, the precise quantitative parameters can differ based on whether the pledget 10 is intended for use by, for example, younger women, older women, women who have given birth, or active women.

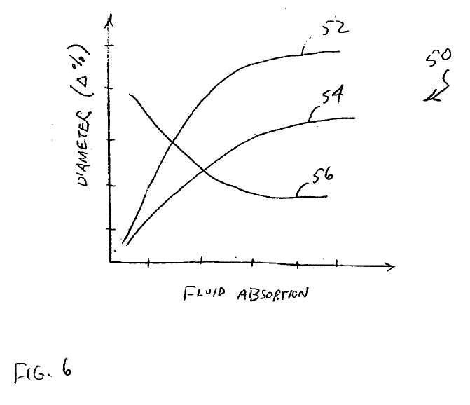

Referring now to FIG. 6, a graphical representation 50 of the density profile illustrates how the various portions of the pledget change during use to anthroprometrically conform to the shape of the vaginal canal. As can be seen by line 52, the top third portion of the pledget increases its diameter during fluid absorption from about 3% to about 200% and preferably from about 30% to about 40%. Line 54 illustrates how the middle third portion of the pledget decreases its diameter. This decrease in diameter is due to “necking,” which can be caused by the expansion of adjacent areas to cause the pulling of material from the middle third portion due to variations in the pore size, distribution, and density or by less fiber being located in the middle third portion to begin with, thereby resulting in less expansion. However, although the middle third portion is shown as decreasing in diameter, the present invention is not limited in this regard and the middle third portion may slightly increase in diameter or stay the same. Line 56 illustrates how the lower third portion of the pledget increases in diameter from about 3% to about 200% and preferably from about 10% to about 20%. The most pronounced expansion of the pledget is preferably in the upper third portion in order to limit the bypass of fluid around the upper third portion to the lower portions. The present invention is not limited in this regard, however, as both the upper third portion and the lower third portion may expand approximately the same amount. All of the foregoing percentages are volume/volume percentages (volume of one liquid component per total volume tampon pledget).

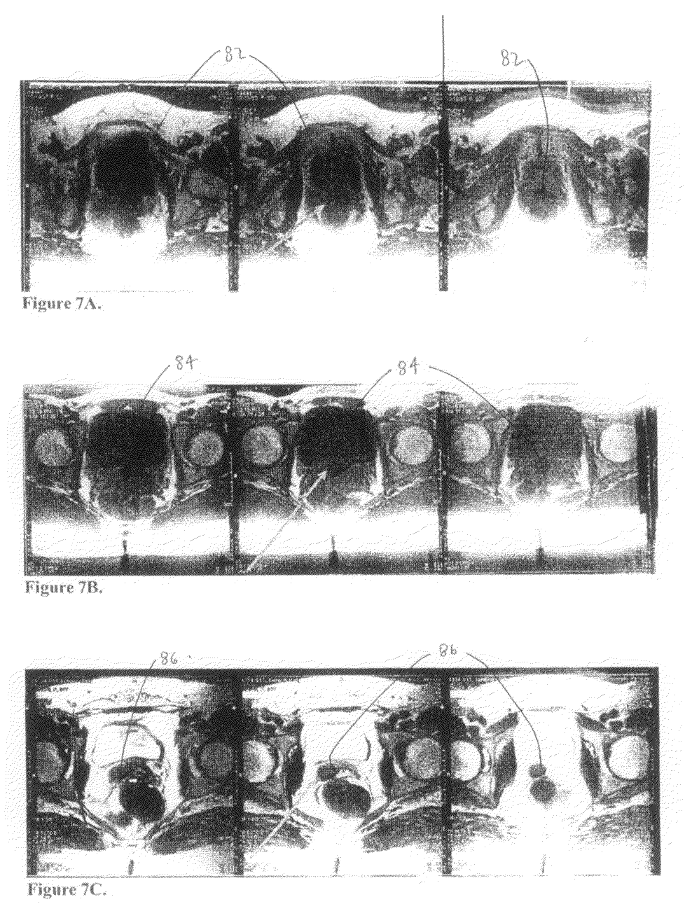

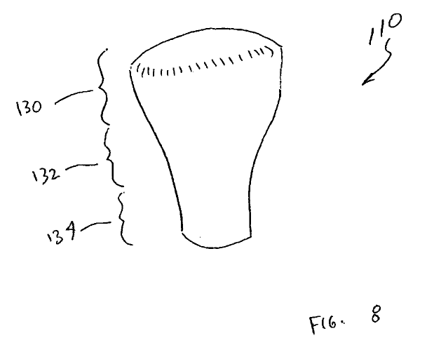

Referring now to FIGS. 7A through 7C, transaxial cross-sectional views of tampon pledgets in vivo are shown for three different women. In FIGS. 7A, 7B, and 7C, tampon pledgets in vivo are shown respectively at 82, 84, and 86. The expansion characteristics for each tampon pledget, which are similar in configuration, are different depending on the shape of the vagina into which the tampon pledget is inserted. During use, tampon pledget 82 is somewhat flattened and conforms to the transverse dimension of the vagina. On the other hand, tampon pledget 84 remains substantially circular in cross-section due to the shape of the vagina of the woman into which that tampon pledget is inserted. Furthermore, tampon pledget 86 approximates an elliptical cross-sectional geometry as a result of the shape of the vagina of the woman into which that tampon pledget is inserted. Such shape changes are influenced by a variety of interdependent factors, namely, the amounts and rates of absorption, the tampon pledget placement in the vagina relative to the source of the bleeding, the cervical os, the amount of pressure exerted by the vaginal muscles, and the particular shape of the vagina.

Based on the foregoing, actual tampon design and geometry (e.g., density profile, initial tampon shape, and the like) suggest that changes to the design and geometry can affect expansion characteristics. Thus, such data supports the fact that the tampon pledgets of the present invention expand approximately into hourglass shapes, as shown in FIG. 2, during use.

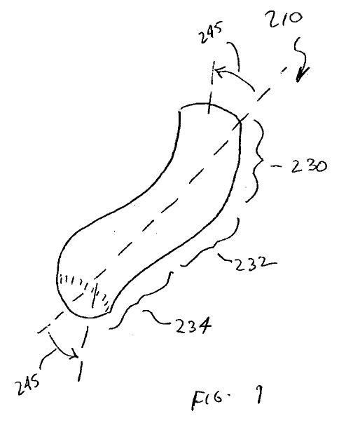

As is shown in FIG. 8, a tampon pledget 110 may be fabricated to have an anthroprometric geometry that approximates a goblet or a cone. In such a device, an upper third portion 130 expands significantly in response to the absorption of menses or body fluids to conform to the shape of the vagina. The middle third portion 132 may experience necking, substantially retain its original diameter, or may slightly increase in diameter (as shown). The lower third portion 134 may also experience necking or substantially retain its original diameter.

As is shown in FIG. 9, a tampon pledget 210 may be fabricated to have an anthroprometric geometry that approximates a serpentine shape. In approximating this serpentine shape, the pledget 210 expands during use and upon contact with fluid to have a slight S-shaped configuration to accommodate a vaginal canal having a substantially corresponding shape (front-to-back). In pledget 210, a middle third portion 232 is slightly smaller in diameter than either a top third portion 230 or a bottom third portion 234. However, the material of the pledget 210 (e.g., rolled or folded web material) can be located such that upon the absorption of fluid, the top third portion 230 and the bottom third portion 234 are pulled in opposite directions, as shown by arrows 245, relative to the middle third portion 232. In the alternative, or additionally, because the material of the pledget 210 often has a “memory” that enables the pledget to “recover” its shape, the S-shaped configuration could be obtained by the process used to manufacture the pledget.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A tampon pledget, comprising:
an absorbent mass of material having a substantially cylindrical geometry;
an insertion end located at one end of said absorbent mass of material;
a withdrawal end located at an opposing end of said absorbent mass of material; and
a withdrawal string extending from said withdrawal end; and
said absorbent mass of material having a density gradient that allows different portions of said absorbent mass to expand anthroprometrically upon the application of fluid to said absorbent mass, thereby allowing said absorbent mass to expand to the shape of a vaginal canal into which said tampon pledget is inserted.

2. The tampon pledget of claim 1, wherein a density of an upper third portion at said insertion end is greater than a density of a middle third portion intermediate said insertion end and said withdrawal end.

3. The tampon pledget of claim 1, wherein a density of a lower third portion at said withdrawal end is greater than a density of a middle third portion intermediate said insertion end and said withdrawal end.

4. The tampon pledget of claim 1, wherein said absorbent mass of material comprises at least one web of absorbent material.

5. The tampon pledget of claim 1, wherein said absorbent mass of material comprises, a first web of material, a second web of absorbent material located at said insertion end, and a third web of absorbent material located at said withdrawal end.

6. The tampon pledget of claim 5, wherein said first web of material and each of said second web of absorbent material and said third web of absorbent material define respective radial density gradients.

7. The tampon pledget of claim 5, wherein said first web, said second web, and said third web are at least one of rolled and folded together.

8. The tampon pledget of claim 1, wherein said absorbent mass of material has a lengthwise density gradient.

9. The tampon pledget of claim 1, further comprising a coverstock located over said absorbent mass.

10. An anthroprometrically expandable tampon pledget, comprising:
an absorbent mass of material, said absorbent mass of material comprising,
a first end having a first density,
a second end having a second density, and
a portion intermediate said first end and said second end, said portion having a third density that is different from at least one of said first density and said second density; and
means for withdrawing said tampon pledget from a vagina into which said tampon pledget is inserted, said means for withdrawing being attached to said absorbent mass of material;
wherein said absorbent mass of material is cylindrical in shape; and
wherein at least said first end of said absorbent mass of material is expandable upon contact with fluid.

11. The anthroprometrically expandable tampon pledget of claim 10, wherein said first end of said absorbent mass of material comprises a first web of said material; and
wherein said second end of said absorbent mass of material comprises a second web of said material.

12. The anthroprometrically expandable tampon pledget of claim 11, wherein said first web of said material is at least one of rolled and folded around a core of material; and
wherein said second rolled web of material is at least one of rolled and folded around said core of material.

13. The anthroprometrically expandable tampon pledget of claim 11, wherein said first web of said material is interwound with a material of said portion intermediate said first end and said second end; and
wherein said second web of said material is interwound with said material of said portion intermediate said first end and said second end.

14. The anthroprometrically expandable tampon pledget of claim 10, wherein said first end, said second end, and said portion intermediate said first end and said second end are compressed into said cylindrical shape.

15. The anthroprometrically expandable tampon pledget of claim 10, wherein said absorbent mass of material is selected from the group consisting of rayon, cellulosic material, and combinations of the foregoing.

16. The anthroprometrically expandable tampon pledget of claim 10, where said first end increases is diameter upon contact with said fluid by about 3% to about 200%.

17. The anthroprometrically expandable tampon pledget of claim 10, where said first end increases in diameter upon contact with said fluid by about 30% to about 40%.

18. The anthroprometrically expandable tampon pledget of claim 10, where said portion intermediate said first end and said second end decreases in diameter upon contact with said fluid.

19. The anthroprometrically expandable tampon pledget of claim 10, where said second end increases in diameter upon contact with said fluid by about 3% to about 200%.

20. The anthroprometrically expandable tampon pledget of claim 10, where said second end increases in diameter upon contact with said fluid by about 10% to about 20%.

21. A method of making an expandable tampon pledget, said method comprising the steps of:
providing at least one absorbent material;
attaching a string to said absorbent material; and
compressing said at least one absorbent material into a final cylindrical shape such that a density gradient is realized over a length of the compressed absorbent material; and
wherein said tampon pledget anthroprometrically expands to the shape of a vagina into which said tampon pledget is inserted upon said absorbent material being contacted with fluid.

22. The method of claim 21, wherein said step of providing at least one absorbent material comprises the step of rolling at least one web into an approximate cylindrical shape.

23. The method of claim 22, wherein said step of rolling at least one web into an approximate cylindrical shape comprises the step of interwinding at least two webs.

24. The method of claim 21, wherein said step of providing at least one absorbent material comprises the step of folding at least one web into an approximate cylindrical shape.

25. The method of claim 21, wherein said step of compressing said at least one absorbent material comprises compressing said absorbent material in at least one of a radial direction and a lengthwise direction.
